(19) United States
(12) Patent Application Publication
(10) Pub. No.: US 2023/0277295 A1
(43) Pub. Date: Sep. 7, 2023

(54) BLOOD FLOW MODIFICATION DEVICE AND METHODS OF USING THE SAME

(71) Applicant: ANAXIOM CORPORATION, Laguna Hills, CA (US)

(72) Inventors: Justin Jerome Ringhofer, Laguna Hills, CA (US); William E. Cohn, Laguna Hills, CA (US); Nick Gentile, Laguna Hills, CA (US)

(21) Appl. No.: 18/040,693

(22) PCT Filed: Aug. 6, 2021
(86) PCT No.: PCT/US2021/045011

§ 371 Date: Feb. 6, 2023

Related U.S. Application Data
(60) Provisional application No. 63/063,652, filed on Aug. 10, 2020.
(60) Provisional application No. 63/149,172, filed on Feb. 12, 2021.

(51) Int. Cl. A61F 2/07; A61F 2/966; A61F 5/00

(52) U.S. Cl. CPC A61F 2/07; A61F 2/966; A61F 5/0013; A61F 2002/068

(57) ABSTRACT

A device for modifying blood flow through a target artery of a patient is described. A method for treating obesity in a patient includes positioning the device in a first artery to decrease blood flow through a second artery causing hypoperfusion to a gastrointestinal organ serviced by the second artery, and interfering with gastrointestinal function to induce weight loss. A system for placement of a blood flow modification device within a first artery to gradually reduce blood flow to a second target artery while maintaining blood flow to the first artery is also provided. The device may be modifiable after treatment to restore or increase blood flow to the artery.

### INCORPORATION BY REFERENCE TO ANY PRIORITY APPLICATIONS

The present application claims priority benefit of U.S. Provisional Application No. 63/063,652, filed Aug. 10, 2020, titled “BLOOD FLOW MODIFICATION DEVICE AND METHODS OF USING THE SAME,” and U.S. Provisional Application No. 63/149,172, filed Feb. 12, 2021, titled “DELIVERY SYSTEM FOR BLOOD FLOW MODIFYING DEVICE AND METHODS OF USING THE SAME,” each of which is hereby incorporated by reference herein.

### BACKGROUND

### Field

An apparatus, and method of using the apparatus, for inducing weight loss in a patient by intentionally modifying blood flow through an artery is provided.

### Description of the Related Art

The number of adults that are obese is exponentially increasing around the world. Obesity is one of the leading causes of death globally.

Current bariatric surgical techniques (e.g., gastric bypass, biliopancreatic diversion) are quite invasive. Many obese adults are not eligible for these surgery because they are at high risk for surgical complications. Thus, there is still a need for bariatric treatments that are less invasive and available to a larger population.

### SUMMARY

The blood flow modification devices described herein may be permanently implanted to gradually modify blood flow through a target artery, for example the celiac artery, to cause hypoperfusion to a gastrointestinal organ serviced by the target artery. The blood flow modification device reduces blood flow to the gastrointestinal organ by at least partially or fully occluding blood flow through the target artery. The reduced blood flow leads to discomfort from overeating. Over time, this results in a reduction of food intake behavior and weight loss. Unlike existing bariatric surgical techniques, the devices and methods described herein are less damaging to the tissue, which leads to fewer complications and faster recovery.

The blood flow modification device does not necessarily occlude the target artery, only the ostium providing access to the target artery. In other words, the blood flow modification device may not be directly placed within the artery in which flow should be reduced. In one example, the blood flow modification device may at least partially occlude the celiac ostium 5, shown in FIG. 1. In this instance, the blood flow modification device can be positioned outside of the celiac artery, for example within the abdominal aorta, to promote occlusion of the ostium of the celiac artery. The blood flow modification may only promote occlusion of the ostium of a single artery, without disturbing flow through other arteries that perfuse the gastrointestinal organ. In other configurations, the blood flow modification device may be positioned within the target artery to at least partially or fully occlude the target artery.

The blood flow modification devices described herein may include a frame, e.g., a tubular stent, supporting a cover. The cover may be polymeric and porous and does not immediately or mechanically occlude the ostium to reduce the likelihood of visceral ischemia. Occlusion takes place over time through endothelialization across the cover. For example, after one month, the ostium may be occluded at least about 20% and/or less than about 100%, for example from 30% to 80%, or from 40% to 70%, such as at least about 60% occlusion, or at least about 70% occlusion.

In use, the blood flow modification device may be percutaneously delivered through the vasculature. A guidewire may be introduced into the femoral artery. A delivery system may be advanced over the guidewire to a first vessel, e.g., abdominal aorta, and the distal tip of the delivery system may be positioned beyond the ostium of the target artery, e.g., celiac artery or other artery perfusing a gastrointestinal organ. Once in position, a first section of the blood flow modification device may be deployed in the first vessel. The first section may be uncovered to facilitate expansion and anchor the device in the first vessel. The cell pattern of the first section may be sufficient to anchor the blood flow modification device without any additional anchors (e.g., barbs or projections). The first section may be the only portion of the blood flow modification device that is uncovered around the entire circumference. Radiopaque markers in the first section may be used to confirm rotational alignment of the device relative to the ostium of the target artery. A cover on a second section the blood flow modification device may be deployed against or across the ostium of the target artery, for example the celiac ostium. The cover may only surround a partial circumference of the device to facilitate expansion of the second section and limit endothelialization to the region surrounding the ostium. No part of the cover fully surrounds any portion of the frame. The cover may extend from the first section to a terminal end of the frame. The cell size in the second section may be larger than the cell size in the first section to limit the amount of foreign material in contact with the aortic wall. Radiopaque markers at a second end of the device may be used to confirm the axial position of the device relative to the target artery.

Over time, it may become desirable to regain access the target artery. The device may have no retrieval mechanism. An instrument, e.g., wire or stylet, may be advanced from the abdominal aorta, through the cover, and into the target artery without removing the blood flow modification device. In other methods, the instrument may be advanced through an un-occluded ostium to a secondary artery, for example the superior mesenteric artery, into the target artery, and through the cover toward the abdominal aorta. The target artery may be accessed by deploying an expandable device, such as a balloon, stent or second blood flow modification device, through the opening created in the sidewall of the blood flow modification device. The amount of blood flow restored through the target artery may be controlled using the expandable device.

Blood flow modification devices for inducing weight loss are described herein. The blood flow modification device may include a frame configured to support a porous cover. The frame may be a tube-like stent. The frame may include a first section at a first end of the frame and a second section at a second end of the frame. The first section may be uncovered and have a first cell pattern. The first cell pattern may be designed to anchor the blood flow modification device in a vessel without any additional anchors or barbs, for example the first cell pattern may have a smaller cell size compared to the second section. The second section may support the cover. The cover only partially surrounds a circumference of the frame. The second section may have a second cell pattern that is different from the first cell pattern. For example, the second section may have a larger cell size to reduce the amount of foreign material against the aortic wall and prevent tearing of the cover. The second section may include a plurality of elongate struts, for example, two, three, four, or more struts. Each strut may be an axial strut extending from the first section to the second end of the frame. The axial struts may be linear without any bends. The cover may extend from one of the plurality of struts to another one of the plurality of struts.

The blood flow modification device may include a frame configured to anchor the blood flow modification device in a first vessel. The frame may include a frame having a first section at a first end of the frame and a second section supporting a porous cover. The porous cover may include a uniform arrangement of openings to permit blood flow through the cover prior to endothelialization. The porous cover may be constructed from a polymeric or non-metal material. The porous cover is configured to be positioned across an ostium of a second vessel. The cover only partially surrounds a circumference of the frame. No portion of frame is fully surrounded by the cover. The cover may extend from an interface between the first section and the second section to a second end of the frame. Only the first section may remain circumferentially uncovered.

The blood flow modification devices described herein may include one or more radiopaque markers. For example, a first subset of radiopaque markers may be in the first section and a second subset of radiopaque markers may be in the second section. The first section may include at least one radiopaque marker configured to be positioned generally opposite and/or generally aligned with the ostium of the target artery and/or the cover. In the first section, one or more radiopaque markers may be positioned at the first end of the device or at the opposite end of the first section, for example between the first section and the second section. In the second section, one or more radiopaque markers may be positioned at the second end of the frame. Each radiopaque marker at the second end may be circumferentially offset or aligned with one of the plurality of struts.

The blood flow modification devices may include a plurality of anchors or attachment points, e.g., eyelets, for attaching the cover, for example using sutures. A first set of anchors may be positioned at the interface between the first section and the second section, and a second set of anchors may be positioned at the second end of the frame. Each anchor at the second end of the frame may be aligned with or circumferentially offset from one of the plurality of elongate struts.

Certain methods of inducing weight loss in a patient are described herein. The method may include advancing a delivery system carrying a blood flow modification device, for example as described herein, to a first vessel (e.g., an abdominal aorta). The method may include deploying the blood flow modification device in the first vessel such that the cover is positioned across the ostium of the target artery. The blood flow modification device may be deployed by retracting a sheath or advancing the device relative to the sheath. When deployed, an end of the device may be positioned between an ostium to the target artery and an adjacent ostium. Over time, blood flow through the ostium of the celiac artery may be gradually occluded. For example, over the three to seven weeks (e.g., one month), blood flow may be occluded by at least 50% and/or less than about 100%, for example at least about 60% or at least about 70%. The device may not fully occlude blood flow through the ostium. Prior to fully deploying the blood flow modification device, rotational alignment of the blood flow modification device may be confirmed, for example using one or more radiopaque markers. For example, in some methods, at least one radiopaque marker may be designed to be positioned opposite the ostium of the target artery.

The blood flow modification device may be rotated to align the cover with an ostium of a target artery (e.g., the celiac artery or other artery perfusing a gastrointestinal organ). This may take place when the entire device is still within the sheath or after only the first section has been deployed. The entire delivery system may be rotated to rotate the device, or the device may be rotated relative to the delivery system. The delivery system may include an external rotational alignment element that is aligned with the cover. The delivery system may be rotated to align the external rotational alignment element with the ostium of the target artery to confirm rotational alignment.

After occlusion of the ostium, it may become desirable to regain access to the ostium. Some methods of re-opening the ostium may include advancing an instrument (e.g., wire, mandrel, or stylet) through the cover and the ostium of the target artery. For example, the method may include advancing the instrument from the first vessel and through the cover to the target artery. In other methods, the instrument may be advanced through a secondary artery and into the target artery, and then through the cover into the first vessel.

A blood flow modification device is provided having a first section with a structural design configured to radially expand and securely attach the device within a first blood vessel. A second section of the device having a second structural design may be configured to provide an initial non-occluding alignment with an ostium of a second vessel. The device design may be configured to substantially retain pre-treatment blood flow rate through the first blood vessel while gradually decreasing blood flow through the second blood vessel to establish collateral blood as endothelial growth reduces the size of the device wall openings to partially or fully occlude the ostium.

The device may include an expandable elongated or cylinder shape, such as a tube comprised of struts and/or elongated connectors forming a network of open cells around the circumference. A top section of the device may have high radial strength for securing the device against a vessel wall, and a lower section provides longitudinal support. The top section may have smaller cells in a higher density cell pattern, such as a diamond pattern, that provide higher radial support. The lower section may have larger cells for alignment with an ostium. Longitudinal stability provided by straight elongated connectors may prevent foreshortening and increase the positional accuracy of the device upon deployment within the artery. The larger cells of the lower section inhibit sudden occlusion when a cell is aligned with an artery ostium and no connectors, or few connectors, overlay or block the ostium.

When deployed and positioned according to methods described herein, a gradual reduction in blood flow through the ostium of the target artery facilitates establishment of collateral blood flow to organs such as the liver, spleen, stomach and/or pancreas. All, or a portion of, the lower section of the device may comprise a material cover over an elongated cell configured to align with the ostium. Endothelial growth reduces blood flow through the cover to partially or fully occlude the ostium restricting blood flow through the target vessel.

Also disclosed herein is a method for restoring blood flow to a blood vessel after a period of time by penetrating the device through a large cell of the lower section, forming an opening adjacent the ostium, inserting an expandable object through the opening, and expanding the object to re-open the large cell and ostium, restoring blood flow to the target artery.

Blood flow modification device designs described herein that have a high cell density top section and a lower cell density bottom section are distinguishable from known endovascular devices, such as stents having uniformly high density strut coverage throughout the device length and a constraining mesh fabric.

Delivery methods for introducing endovascular devices, such as stents, within a body lumen may employ radiopaque markers to facilitate placement of the stent in the required vessel segment. In some scenarios, radiopaque markers are insufficiently visible to adequately ensure a desired placement of a device within the body.

A system is described for delivering and positioning a blood flow modification device within a body lumen of a patient. A delivery system may include an external rotational alignment element to facilitate alignment of the blood flow modification device within the body lumen.

A two-part blood flow modification device configured to be placed in a body lumen of a patient is also described. The device may include a tube-shaped member that radially expands to accommodate variations in the diameter of an artery in which it is deployed, and/or to reduce the number of device sizes required to treat a majority of the patient population. The blood flow modification device may include a frame, such as a stent, that is partially covered by a cover or mesh to promote endothelial growth. The frame may include a radially expandable portion that is not covered by the cover or mesh, and a restrained portion that is covered by the cover or mesh. A covered portion may be configured to align with a target artery ostium. Alignment is facilitated by the delivery system described herein.

A device delivery system may have a rotational alignment element that is external to the body of the patient throughout the deployment process. A hemostasis valve “Y” connector located at a proximal end of a catheter and that is held by the physician during use, is fixed in place to prevent rotation around the longitudinal axis of the delivery system forming the external rotation alignment element. A method is described for deploying the blood flow modification device within a body lumen, and rotationally aligning the device by referencing the position of the rotational alignment element of the delivery system. The external rotational alignment element aligns the blood flow modification device with the ostium of a target body lumen. A pin pull delivery system may be used in the methods described herein.

The blood flow modification device frame may comprise a shape memory material (e.g., a metal alloy frame). A material such as a mesh may cover all or at least a portion of the circumference of the frame and all or at least a portion of the length of the frame. The frame may include a covering in which a portion of the circumference of the frame remains uncovered, may radially expand between approximately 7 mm to 25 mm.

A method is provide in which the blood flow modification device is loaded into the delivery system in an orientation that aligns with the external rotational alignment element of the delivery devise. Both the blood flow modification device and external rotational alignment element may be configured to be aligned in an upwardly facing direction when inserted in the body for delivery.

When deployed in a first body lumen, the blood flow modification device may be configured to be aligned in an initial non-occluding alignment relative to an ostium of a second blood vessel. When aligned with an ostium of a second vessel, the device may be configured to substantially retain a pre-treatment blood flow rate through the first blood vessel in which it was deployed while gradually decreasing blood flow through the second, target blood vessel and thereby restricting blood flow to an organ supplied by the target blood vessel. Collateral blood flow may be gradually established as endothelial growth begins to occlude holes or opening on the material covering the device frame, reducing the size of openings to partially or fully occlude the ostium of the second vessel.

The delivery system and blood flow modification device described here may be used to treat obesity by at least partially restricting blood flow to a gastrointestinal organ serviced by the at least partially occluded artery. Thus, a method for treating obesity in a patient is provided that includes providing a delivery system having an external rotational alignment element, positioning a blood flow modification device in a first artery to decrease blood flow through a second artery, causing hypoperfusion to a gastrointestinal organ serviced by the second artery and interfering with gastrointestinal function to induce weight loss. By methods and devices provided herein, gradual partial or full occlusion of the target artery lumen allows for collateral blood flow to be established.

### BRIEF DESCRIPTION OF THE DRAWINGS

These and other objects and features of the present invention will be more fully disclosed by the following detailed description, which is to be considered with the accompanying drawings wherein like numbers refer to like parts, and further wherein:

FIG. 1 is a schematic view showing locations of arteries of a human body.

FIG. 2 illustrates a delivery device and blood flow modification device deployed within a human body.

FIG. 3 depicts a flat view of a blood flow modification device design having first and second cell patterns.

FIG. 4 depicts a blood flow modification device comprising a cover to promote endothelial growth.

FIG. 5A depicts a cross-sectional view showing placement of a blood flow modification device occluding an artery ostium.

FIG. 5B depicts a top-down view of the device placement illustrated in FIG. 5A.

FIG. 5C depicts a top-down view showing orientation of a device occluding an artery ostium.

FIG. 6 depicts a flat view of another blood flow modification device relative to an artery ostium.

FIG. 7 depicts a flat view of yet another blood flow modification device relative to an artery ostium.

FIG. 8 depicts a flat view of yet another blood flow modification device.

FIG. 9A depicts a flat view of yet another blood flow modification device.

FIG. 9B depicts an enlarged portion of the embodiment of FIG. 9A.

FIGS. 10A and 10B depict yet another blood flow modification device that may be positioned within the target artery.

FIGS. 11A-11D depict different views a blood flow modification device.

FIG. 12A depicts the blood flow modification device frame shown in FIG. 11A.

FIG. 12B is a flat view of the blood flow modification device frame shown in FIG. 12A.

FIG. 13 illustrates a cover of a blood flow modification device shown in FIG. 11A.

FIG. 14A illustrates a location of a celiac artery and SMA relative to a flat version of the blood flow modification device shown in FIG. 11A.

FIG. 14B illustrates a schematic cross-section of the blood flow modification device of FIG. 11A relative to an artery ostium.

FIG. 15 depicts an illustration of a blood flow modification device delivery system having an external rotational alignment feature, and further depicting the blood flow modification device in FIG. 11A.

FIG. 16A depicts a hemostasis valve Y connector as an external rotational alignment element rotationally aligned with a blood flow modification device.

FIG. 16B illustrates a blood flow modification device partially deployed from a delivery system device.

FIGS. 17 and 18 illustrate a delivery system and blood flow modification device for use within a human body.

### DESCRIPTION

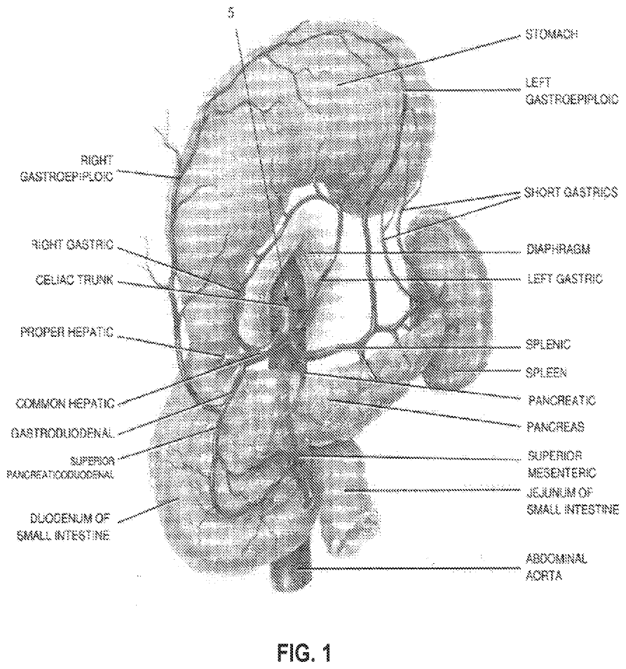

The present disclosure is directed toward methods and devices for inducing weight loss. The method may include deploying a blood flow modification device within a first artery and positioning the device adjacent the ostium of a second target artery that supplies blood to an organ of the gastrointestinal tract of the patient. The method may further include gradually reducing blood flow through the target artery as endothelial growth decreases the size of openings of a portion of the device cover overlaying the target artery ostium, thereby restricting blood flow to one or more organs and inducing weight loss. Hypoperfusion of the stomach or other gastrointestinal organ may be effected by modification of one or more target blood vessel of the gastrointestinal tract at an anatomical location shown in FIG. 1, which may be the celiac artery or superior mesenteric artery; in other embodiments, the blood vessel may be the left gastric artery, the right gastric artery, the left gastroepiploic artery, the right gastroepiploic artery or the common hepatic artery, the inferior pancreaticoduodenal artery, jejunal and/or ileal arteries, or a combination thereof.

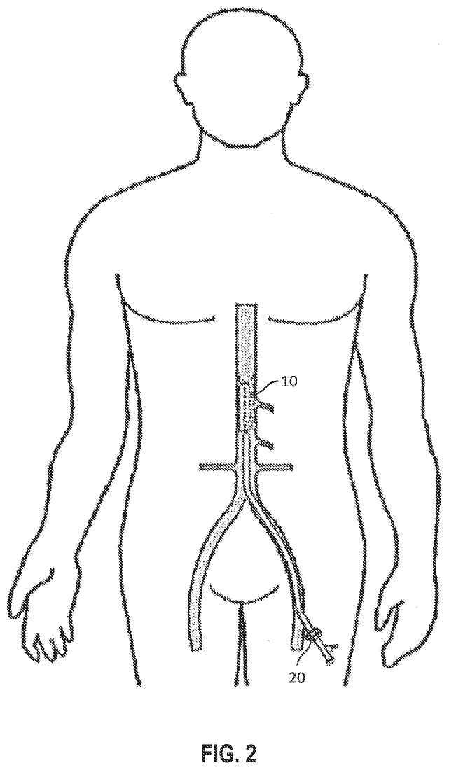

With reference to FIG. 2, blood flow modification device 10 may be deployed from a delivery system 20 within the abdominal aorta covering the ostium of the celiac artery, and upon endothelialization of the device cover, blood flow through the celiac artery to the stomach, is gradually, partially or fully occluded causing weight loss. removed. As shown, the blood flow modification device 10 covers the ostium of the celiac artery without obstructing flow to any other arteries, for example the superior mesenteric artery.

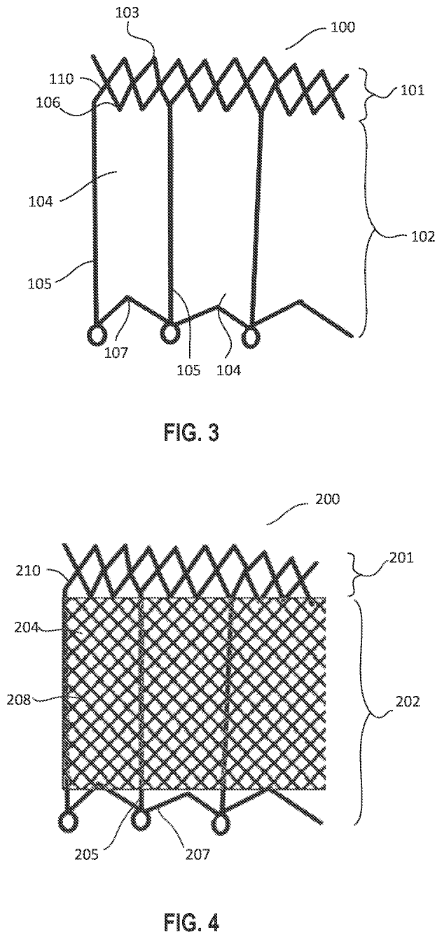

In FIG. 3, a flat view a blood flow modification device 100 is provided wherein the frame 110 comprises more than one cell pattern throughout the device length. In FIG. 1, the frame 110 comprises a first section 101 with a dense population of cells 103, and an adjacent second section 102 having a lower number of larger cells 104. An exemplary first section 101 may comprise short interconnected struts forming, for example, a dense diamond pattern of cells 103 that are smaller than the cells of the second section. The dense cell pattern of the first section 101 may be configured to provide high radial strength to facilitate locking the device in place against the artery wall and preventing migration after placement.

The second section 102 may comprise a plurality of large elongated cells 104 formed between elongated connectors 105. Straight, elongated connectors prevent foreshortening of the device and increase the accuracy of the deployment position. Spacing between adjacent connectors 105 may be configured to form a cell 104 that covers an ostium without an overlaying connector 105. A plurality of straight elongated connectors 105, are depicted without bends or curves for the length of the second section 102. Connectors 105 may be substantially normal to top and bottom ends of the second section 102. Alternatively, one or more connectors 105 may be slanted between top and bottom ends of the second section 102. The length of the connector 105 may be the same as or greater than the second section 102. Adjacent connectors 105 may be parallel or obliquely arranged. Elongated connectors 105 may be attached to support edges at top and/or bottom ends.

Support edges comprise struts 106, 107 configured, for example, in a triangular, or crown-shaped pattern. Support edges may form the top and /or bottom ends of the elongated cells 104.

To reduce the size of the device 100 for insertion in a delivery device or vessel, elongated connectors 105 and/or struts may comprise a shape recovery material that returns the device 100 to a pre-deformed shape after deployment. A shape recovery material may have a thermal expansion memory at blood temperature. Materials include, but are not limited to, a polymer, a metal, or metal alloy, including a nickel and titanium alloy such as nitinol. Suitable materials may also comprise a metal, or a metal alloy, that are expandable using a balloon or other devices commonly known for use in expanding endovascular stents.

The frame 110 made from shape recovery material may be made by techniques known for use in manufacturing endovascular stents, and the frame 110 may comprise laser cut slotted tubes, multilink hoops and sinusoidal continuous wire, and may be formed as a single unit that is wound, folded and welded into shape.

Blood flow modification devices may be the frame alone or further comprise a material cover. In FIG. 4, an exemplary two-part device 200 is depicted having a cover 208 overlaying the lower section 202 of the frame 210, including any of the features of the frame 110 shown in FIG. 3, to modify blood flow to a target artery. The cover 208 overlaying all or a portion of the large cells 204 of the lower section 202 may be connected to elongated connectors 205 and/or the support edge 207, for example, by stitching. A sufficient number of connectors 205 is provided to hold the cover 208 against the artery wall and to prevent migration when deployed. A region of the device configured to cover an ostium has no elongated connectors 205, or a reduced number of connectors 205.

Expansion of the uncovered top section 201 may be greater than the covered or partially covered lower section 202. Where a cover 208 overlays a partial or entire circumference of the device 200, expansion of the lower section 202 upon deployment may be constrained by the dimensions allowed by the cover 208. In some embodiments, at least a portion of the circumference is uncovered, allowing unconstrained portion of the device 200 to continue expanding. Partially covered devices comprise expansion ranges compatible with a range of blood vessel diameters, reducing the number of product sizes necessary to ensure a good fit.

A cover 208 is provided over elongated connectors 205 and at least one elongated cell 204 to cover the ostium. For example, at least two-thirds of a circumference of the device 200 is covered to ensure the cover 208 is placed over the ostium of a second target artery when deployed in the first artery. The cover 208 comprises openings or holes so that the rate at which blood flowing through the length of the device in the first artery may be controlled as it flows through the cover and into the second target artery. The size of the openings may be selected to have minimal impact on flow rate through the target artery initially upon deployment. As endothelial cell growth gradually reduces the size of the openings, blood flow rate is gradually reduced. Thus, the size of the openings may be selected to control the rate at which endothelial cell growth reduces the size of the openings, and consequently, the rate at which blood flow through the cover and the target artery ostium is reduced. Over time, while blood flow through the length of the device is retained, endothelial growth may fully occlude blood from flowing through the cover, thereby blocking blood flow through the target artery. In other embodiments, endothelial growth does not fully occlude the openings, retaining some blood flow through the cover and target artery.

The cover 208 may be designed to attract tissue regrowth, such as a vascular patch material. The cover material may comprise, but is not limited to, microporous polyester-urethane, polyester, PTFE (polytetrafluoroethylene), ePTFE (expanded polytetrafluoroethylene), or PP (polypropylene), and may be a woven or unwoven fabric, mesh, or film. For example, the cover 208 may comprise polyester woven fabric having a nominal thickness of about 0.25 mm (Bard® DeBakey® woven fabric #007956, Bard Peripheral Vascular, Inc. Tempe Ariz.). The cover 208 may initially block up to 25% of the ostium, or between 0% and 25%, or between 5% and 20%, of the ostium prior to endothelial growth. Openings may be approximately in the range of 1 mm×1 mm (or 1 mm diameter) to about 5 mm×5 mm (or about 5 mm in diameter), such as approximately 2 mm×4 mm, or approximately 3 mm×3 mm (or 3 mm in diameter), or approximately 4 mm×4 mm (or 4 mm in diameter). Holes may be formed, for example, by puncturing or cutting a fabric or film, to achieve a desired size, and in some embodiments, the material width between holes may be approximately 0.2 mm to approximately 2 mm. The cover 208 may be selected to provide at least partial occlusion from endothelial growth over, for example, three to seven week period such as a one-month period. In some embodiments, the approximate average percent occlusion of the openings after a treatment period (e.g., about 1 month) may be from 20% to 100%, or from 30% to 80%, or from 40% to 70%, such as at least 50% occlusion, at least 55% occlusion, at least about 60% occlusion, or at least about 70% occlusion.

A method is provided for positioning a blood flow modification device 100, 200 in a first blood vessel to modify blood flow through a second blood vessel. In some embodiments, blood flow through the first blood vessel in which the device 100, 200 is deployed is substantially unaffected while the device 100, 200 is in place, while a blood flow through a second blood vessels is gradually reduced. Modification of blood flow through a second blood vessel may be selected to induce mesenteric ischemia, impeding normal gastrointestinal functioning to induce weight loss. When placed in a first artery adjacent the ostium of a second artery, the device may gradually reduce blood flow through the second artery that supplies to an organ of the gastrointestinal tract while collateral blood flow to the organ is established. The second artery may comprise the celiac artery resulting in perfusion of one or more of the liver, spleen, stomach or pancreas, or the superior mesenteric artery, or one or more of the branches thereof.

A device 300 and a method of positioning a device within a blood vessel is illustrated in FIGS. 3A through 3C. The device 300 may include any of the features of the device 100, 200. An exemplary device 300 is deployed in a first artery 320, such as the abdominal aorta, and the top section 301 is positioned above the ostium 322 of a second target artery 324, such as the celiac artery. Optionally, the device 300 may include one or more anchors 311, for example barbs, on the top section 301 lock the device in place within the first artery and the radial strength of the expanded top section prevents migration after deploying. The lower section 302 of the device 300 may be configured to have a larger cell 304, compared to cells in the top section 301, covering the ostium 322 of the target artery 324 when deployed. In the exemplary embodiment of FIG. 3B, to facilitate a preferred orientation of the device relative the target artery, elongated connectors (305, 305′ and 305″) are spaced substantially equally around the device 300 circumference, and opposite open cells 304, 304′ and 304″ (e.g., having no elongated connectors). As illustrated in FIG. 3C, a method comprises placing the device within the first artery 320 (e.g., the abdominal aorta), and orienting a first elongated connector 305 against the artery wall directly opposite the target artery ostium 308 (e.g., celiac artery ostium), so that the cell 304 opposite the first elongated connector 305 is aligned over the target artery ostium 308. Other positions of the elongated connectors 305 within the first vessel 320, for example, the device 300 may be aligned such that an open cell 304 between the struts 305 is positioned opposite the ostium 322.

One or more radiopaque (RO) markers 309 may be provided to assist the physician with visualizing the position the device in the blood vessel. An eyelet may be formed on a bottom end of an elongated connector 305 and may be filled with radiopaque material, such as a gold-containing material or a tantalum-containing material. As illustrated in FIGS. 3A through 3C, visually aligning an RO marker 309 (located on an elongated connector 305) against a vessel wall that is opposite the target ostium 308, may be used to confirm placement of a large opposing cell 304 over the ostium 308 and the absence of an elongated connector in front of the ostium 308, or vice versa.

A method for deploying an expandable blood flow modification device 300 (or any of the other devices described herein) comprises retracting a sheath to unsheathe the proximal end of the device 300 to partially expand the denser cell pattern of the top section 301; confirming the device deployment location with the partially expanded cell pattern; further retracting the sheath allowing the top section 301 to fully expand and locking the device at the location in the vessel; and/or retracting the sheath to unsheathe the distal end of the expandable device 300, allowing the lower section 302 to fully expand.

The dimensions of the device 300 (or any of the devices described herein) may be sufficiently sized to be retained within a range of artery sizes; for example, the device 300 may form a tube having an outer diameter that may expand (before over expansion) to a diameter between 2 mm and 30 mm, or between 7 mm and 25 mm, or between 10 mm and 30 mm, such as 13 mm to 30 mm, such as approximately 21 mm, or between 2 mm to 10 mm, such as between 2 mm to 5 mm. The length of the device in a deployed state may be determined by the target location, wherein the length of a top section 301 may be sufficient to hold the device 300 in place within the artery, and the length of the lower section 302 may be sufficiently long to cover an ostium of a branching artery without blocking any adjacent branching arteries. For example, in some embodiments, the total device length may be 50 mm or greater, such as between 50 mm and 55 mm, for example 54 mm, or the length of the device may be 50 mm or less, such as between 30 mm and 50 mm, or between 20 mm and 30 mm, or between 10 mm and 30 mm, The device top section 301 may comprise approximately 10% to 30% of the total device length, such as from 15% to 25% (e.g., approximately 20%) of the length of the device. The lower section 302 of the device may comprise approximately 70% to 90% of the total device length, such as approximately from 75% to 85% of the device length (e.g., approximately 80% of the device length). In some embodiments, the length of an elongated connector may be from 25 mm to 55 mm, or from 30 mm to 50 mm, or from 35 mm to 45 mm, or 20 mm to 45 mm. or from 7 mm to 30 mm.

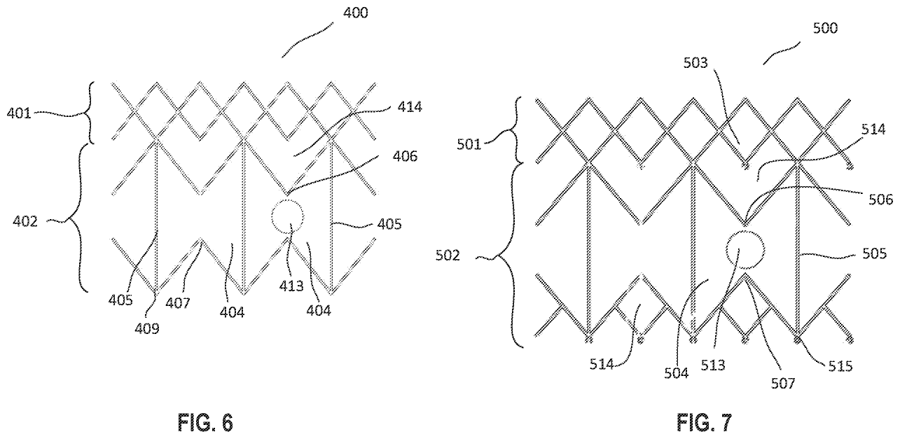

Additional blood flow modification devices are illustrated in FIGS. 6 through 9B. For illustrative purposes to provide an unobstructed view of the cell patterns, the devices are shown uncovered, though a cover may be provided as described herein. Any of the frames of the blood flow modification devices may include any combination of features described herein.

Exemplified in FIG. 6, a flat view of a blood flow modification device 400 comprising a top section 401 having a diamond pattern with a higher cell density than the lower section 402 is illustrated. In the lower section 402, a plurality of top and bottom triangle support struts 406, 407 and elongated connectors 405, define a plurality of open cells 404. A location 413 for covering a target ostium (e.g., a celiac artery ostium) within a cell 404 is identified. Triangular cells 414 formed between the top section and elongated lower cells 404 may provide stability to the device. Eyelets may be incorporated to form radiopaque markers 409, cover attachments, and/or anchors on one or more elongated connectors 405. A cover (not depicted) may overlay at least a portion of the lower section 402 configured to align with the target ostium, and anchored to the device at an eyelet.

In FIG. 7, a device 500 may include a lower section 502 having a plurality of elongated connectors 505 connected by top and bottom support edges form by triangle support struts 506, 507. A row of open triangular cells 514 may be formed between the top section 501 higher density diamond cell pattern 503 and open elongated cells 504. An area 513 free from connectors 505 may be configured to cover a target ostium. The lower section 502 may be partially covered by a material (not shown) that is radially supported by the elongated connectors 505, triangle supporting struts 506, 507 and/or triangular cells 514. One or more diamond-shaped cells 514 may be provided at the bottom end of the device 500 to provide further support and anchoring for the material cover. Anchor points 515, for example eyelets, may be used for attaching the cover (e.g., by stitching) to the device, and one or more eyelets aligned with elongated connectors 505 may be filled with radiopaque material to form radiopaque markers.

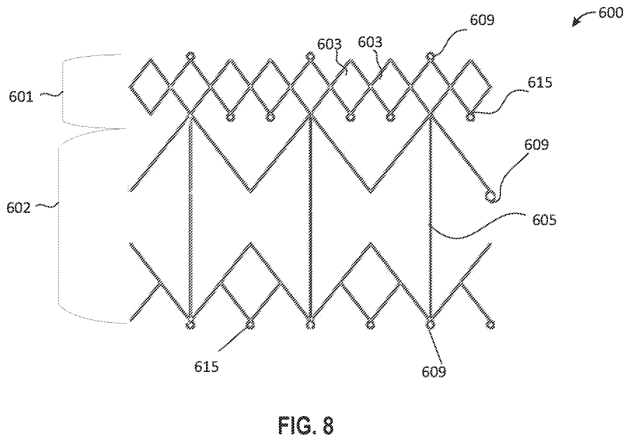

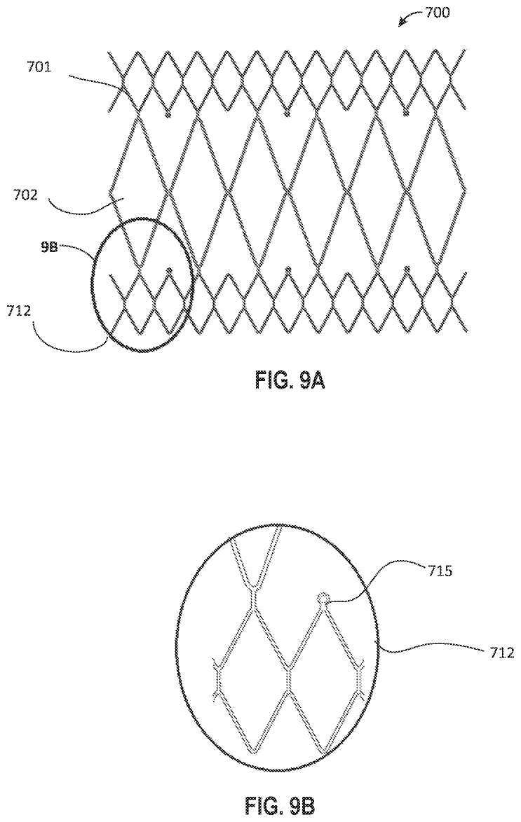

In FIG. 8, a device 600 may include a top section 601 having a greater number of cells 603 between elongated connectors 605 of the lower section 602, compared to the embodiments of FIGS. 6 and 7. A radiopaque marker 609 may be provided at a top end to ensure placement of the proximal, or top, section 601 of the device within the first artery is above the target artery. Optionally, a large radiopaque marker 609 within an open cell of the lower section 602 facilitates alignment of the cell over a target ostium. The bottom device end may comprise a radiopaque marker 609 to ensure the device 600 is above a third artery located below the target artery. For example, where the first artery is the abdominal aorta and the second target artery is the celiac artery, the bottom radiopaque marker 609 may ensure the device 600 does not block the superior mesenteric artery (SMA). One or more anchors 615 may be provided for attaching a cover on the lower section 602. The cover may be stitched to one or more eyelets preventing the cover from migrating inward to the center of the tube-shaped. The cover may be retained against the artery wall by attachment to eyelets, elongated connectors and/or struts, such as the triangle support struts.

In FIGS. 9A and 9B, a blood flow modification device 700 comprises a proximal first section 701 and a distal second section 712, each comprising a high cell density pattern for radial strength and anchoring at proximal and distal ends. A middle section 702 located between the top and bottom sections 701, 712 comprises a lower density cell pattern formed by, for example, oblique, diagonal, or zigzag elongated connectors. A cover (not shown) may overlay at least a portion of the cells of the middle section 702, and optionally, the second section 712. A diamond pattern of the second section 712 provides additional support to the cover, and eyelets 715 may be provided for anchoring the cover or forming radiopaque markers. The length of the middle section 702 between top and bottom sections 701, 712 may comprise from about 50% to about 80% of the total device length.

A method is provided for reopening the target artery (such as the celiac artery) after placement of any of the devices described herein and/or endothelial growth. The method may include forming an opening in a cell of a device side wall by pushing an instrument (e.g., stylet or wire) through endothelial growth formed on an area of a cover that overlays the ostium. The cover material may be stretchable to accommodate an expandable device through the sidewall opening. The target artery may be accessible by pushing an expandable device such as a balloon, stent or second blood flow modification device through the opening in the device side wall and expanding it. The cover, elongated connectors, and/or endothelial growth may be pushed aside by the expandable device (e.g., second device, balloon or stent), reopening the artery ostium and/or the target artery, restoring blood flow.

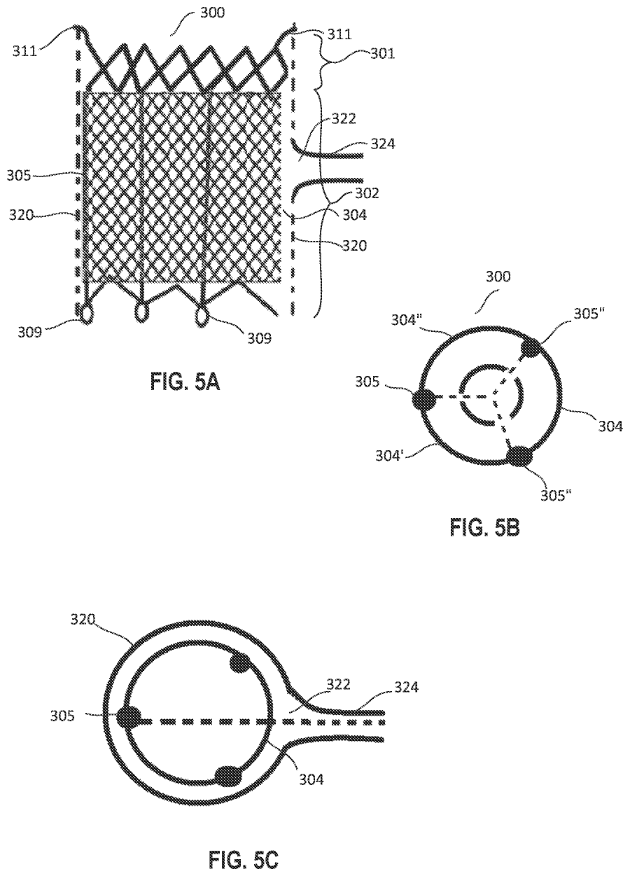

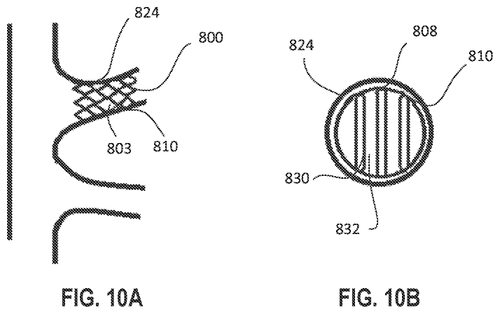

FIG. 10A illustrates a cross-sectional view of a blood flow modification device 800 deployed within a target artery 824, rather than covering the ostium of the target artery (as depicted in FIGS. 5A-5C). An expandable device frame 810 having a tube structure comprises a plurality of cells 803 that hold the device 800 against the artery wall. In the top-down view of FIG. 8B. The device further comprises a cover 808 located across a lumen, for example over the top end, of the tubular frame 810 to reduce blood flow through the top end of the device 800, impeding blood flow through the length of the device and through the target artery 824 in which the device 800 is deployed. The cover 808 may include material threads 830 and openings 832 therebetween. Blood flow rate through the tube structure may be reduced after endothelial cell growth (not shown) reduces the size of the openings between threads and at least partially occludes the cover 808. After a treatment period, blood flow may be restored through the device and into the artery by forming an opening through the at least partially occluded cover 808, and inserting a second expandable device to push away the endothelial growth and/or threads of the cover.

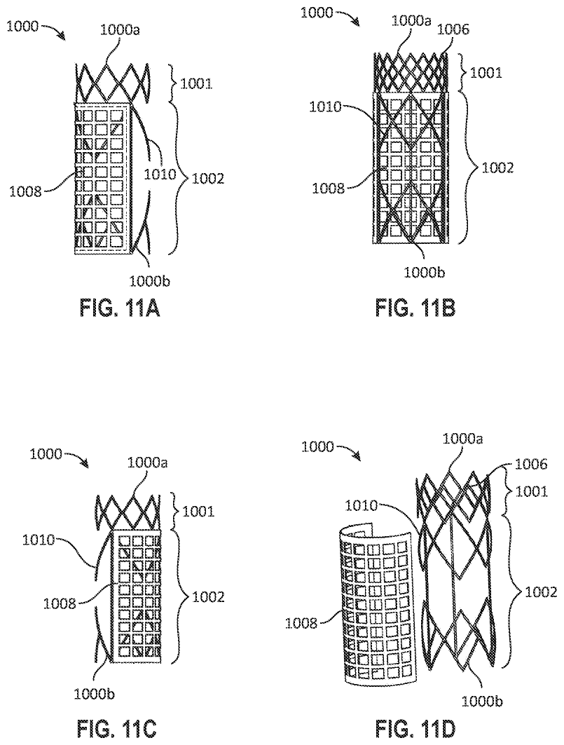

FIGS. 11A-11D illustrate another blood flow modification device 1000 that may include any of the features of the above-described devices or any of the benefits described above. Numerals used to identify features of the blood flow modification device 1000 are incremented by factors of one hundred (100) to identify like features of the earlier described embodiments. This numbering convention generally applies to all of the figures. Any component or step disclosed in any embodiment in this specification can be used in other embodiments.

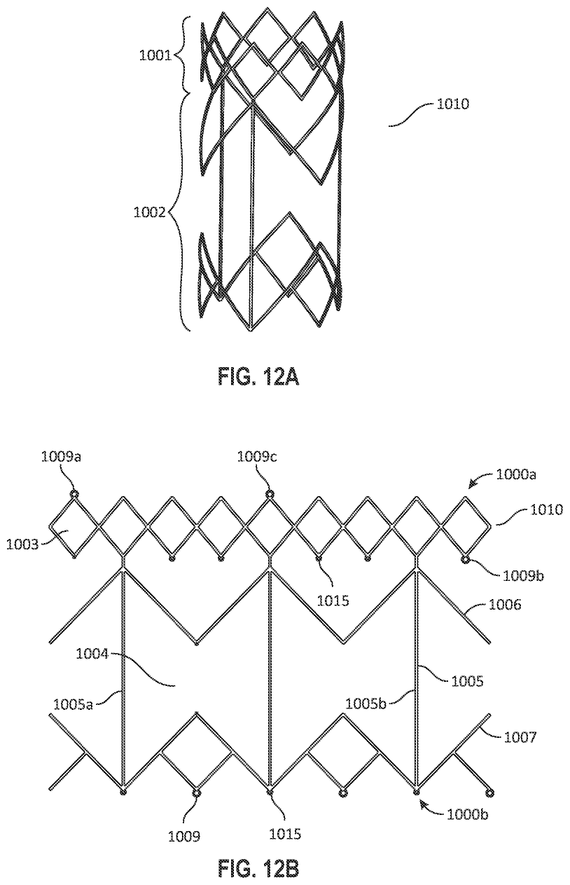

The blood flow modification device 1000 may comprise a frame 1010 and a frame cover 1008. The frame cover 1008 overlays at least a portion of the frame length and circumference. The frame cover 1008 may be attached to the frame 1010 at attachment points 1015 (shown in FIG. 12B), for example, by stitching. The frame cover 1008 may comprise a fabric or mesh, such as a polyester mesh, having openings that supports endothelial growth.

The cover 1008 may only extend a partial circumference of the frame 1010. The cover 1008 may extend less than 360 degrees around a circumference of the frame 1010, for example, at least about 60 degrees and/or less than or equal to about 300 degrees around the circumference, at least about 90 degrees and/or less than or equal to about 270 degrees, at least about 120 degrees and/or less than or equal to about 240 degrees around the circumference of the frame, or at least about 180 around the circumference of the frame. The cover 1008 may extend only partially along an axial length of the frame 1010, but at least a majority of an axial length of the frame 1010.

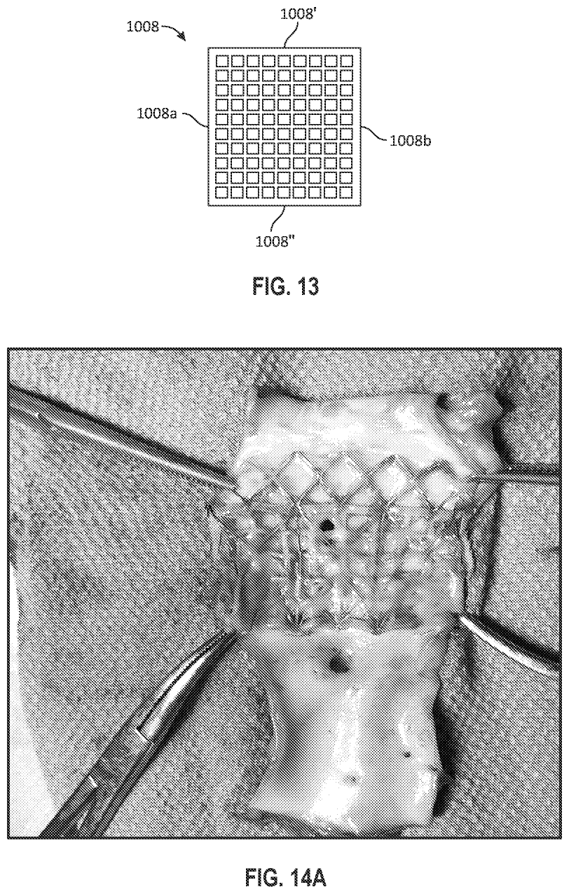

As shown in FIG. 13, the cover 1008 may include a first lateral edge 1008a and a second lateral edge 1008b. When applied to the frame 1010, the entire first lateral edge 1008a is spaced apart from the entire second lateral edge 1008b such that no axial portion of the frame 1010 is fully surrounded by the cover 1008.

The device 1000 may include a first section 1001 that remains uncovered for anchoring and a second section 1002 that is at least partially covered by the cover 1008. The first section 1001 may be positioned at a first end 1000a of the device 1000. The first section 1001 may be the only axial portion completely uncovered. The second section 1002 may have a length that is at least 2×, at least 3×, or at least 4× a length of the first section 1001. The cover 1008 may extend from an interface between the first and second sections 1001, 1002 to the second end 1000b. A top edge 1008′ of the cover 1008 may be positioned at the bottom of the first section 1001, and a bottom edge 1008″ of the cover 1008 may be positioned at the terminal end 1000b of the device, see FIGS. 11A-11D, 13. The cover 1008 extends only a partial axial length of the frame 1010. Although not shown, the device 1000 may have an additional anchoring section similar to the first section 1001 at the second end 1000b of the device, such that the second section 1002 is positioned between the first section 1001 and the additional anchoring section. The additional anchoring section may remain uncovered or be at least partially covered by the cover 1008.

As shown in FIG. 13, The cover 1008 may include openings or holes so that the rate at which blood flows through a sidewall of the device 1000 may be controlled. The size of the openings may be selected to have minimal impact on flow rate through the target artery initially upon deployment. As endothelial cell growth gradually reduces the size of the openings, blood flow rate through the cover 1008 may be gradually reduced. The size of the openings may be selected to control the rate at which endothelial cell growth reduces the size of the openings, and consequently, the rate at which blood flow through the cover 1008 and the target artery ostium is reduced. Over time, while blood flow through the length of the device 1000 is retained, endothelial growth may fully occlude blood from flowing through the cover 1008, thereby blocking blood flow through the target artery. In other embodiments, endothelial growth does not fully occlude the openings, retaining some blood flow through the cover 1008 and target artery.

The cover material may comprise, but is not limited to, microporous polyester-urethane, polyester, PTFE (polytetrafluoroethylene), ePTFE (expanded polytetrafluoroethylene), or PP (polypropylene), and may be a woven or unwoven fabric, mesh, or film. In embodiment, the cover comprises polyester woven fabric having a nominal thickness of about 0.25 mm (Bard® DeBakey® woven fabric #007956, Bard Peripheral Vascular, Inc. Tempe Ariz.). The cover may initially block up to 25% of the ostium, or between 0% and 25%, or between 5% and 20%, of the ostium prior to endothelial growth.

The cover 1008 does not immediately occlude the ostium. The cover 1008 may have a square or rectangular shape with a uniform porosity to enable blood flow, at least initially. For example, the cover 1008 may have rows of similarly sized openings. Each row may have at least about five openings and/or less than or equal to about 15 openings, for example ten openings. There may be at least about five rows and/or less than or equal to about 15 rows of openings. The number of rows may be the same or different from the number of openings in each row. The cover 1008 may be surrounded by a border without any openings.

Openings may be approximately in the range of 1 mm×1 mm (or 1 mm diameter) to about 5 mm×5 mm (or about 5 mm in diameter), such as approximately 2 mm×4 mm, or approximately 3 mm×3 mm (or 3 mm in diameter), or approximately 4 mm×4 mm (or 4 mm in diameter). Holes may be formed, for example, by puncturing or cutting a fabric or film, to achieve a desired size, and in some embodiments, the material width between holes may be approximately 0.2 mm to approximately 2 mm. The cover may be selected to provide at least partial occlusion from endothelial growth over, for example, a one-month period. In some embodiments, the approximate average percent occlusion of the openings after a treatment period (e.g., about 1 month) may be from 20% to 100%, or from 30% to 80%, or from 40% to 70%, such as at least 50% occlusion, at least 55% occlusion, at least 60% occlusion, or at least 70% occlusion.

The frame 1010 may comprise a metal or metal alloy stent, having a tube-shaped body, as exemplified in FIG. 12A. When located on the outer surface of the frame 1010, the cover 1008 may be oriented between the wall of the body lumen wall in which it is deployed and the frame 1010. To reduce the size of a device for insertion in a delivery device or vessel, the frame 1010 may comprise a shape recovery material that returns the device to a pre-deformed shape after deployment. A shape recovery material may have a thermal expansion memory at blood temperature. Materials include, but are not limited to, a polymer, a metal, or metal alloy, including a nickel and titanium alloy such as nitinol. Suitable materials may also comprise a metal, or a metal alloy, that are expandable using a balloon or other devices commonly known for use in expanding endovascular stents.

In the illustrations of FIGS. 12A and 12B, a tube-shaped view and a flat view of an exemplary embodiment of a frame 1010 is provided wherein the device comprises more than one cell pattern throughout the device length. A first section 1001 is depicted having a dense population of cells 1003, and an adjacent second section 1002 having a lower number of larger cells 1004. An exemplary first section 1001 may comprise short, interconnected struts forming, for example, a dense diamond pattern of cells 1003 that are smaller than the cells 1004 of the second section 1002. The dense cell pattern may be configured to provide quick expansion and high radial strength to facilitate locking the device in place against the artery wall and preventing migration after placement.

As shown in FIG. 12B, the first section may include a single ring of cells 1003 at the first end 1000a. The cells 1003 may be diamond-shaped and uniform in size. The cell pattern of the first section 1001 may anchor the device 1000 without any additional anchors, such as barbs or projections, but it is envisioned that additional anchors could be provided at one or both ends of the device 1000 to facilitate anchoring.

The second section 1002 may comprise a plurality of large elongated cells 1004 formed between elongated connectors or struts 1005. Straight, elongated connectors 1005 may prevent foreshortening of the device and increase the accuracy of the deployment position. The larger cells 1004 also reduce the stress on the cover 1008 during device expansion to prevent the cover 1008 from tearing. Spacing between adjacent connectors 1005 may be configured to form a cell 1004 that covers an ostium without an overlaying connector 1005, although in some configurations, one of the connectors 1005 may overlay the ostium. A plurality of straight elongated connectors 1005, for example three connectors 1005, are depicted without bends or curves for the length of the second section. Connectors 1005 may be substantially normal to top and bottom ends of the second section 1002. Alternatively, one or more connectors may be slanted between top and bottom ends of the second section. Adjacent connectors may be parallel or obliquely arranged. Elongated connectors 1005 may be attached to support edges 1006, 1007 at top and/or bottom ends. Support edges 1006, 1007 comprise struts configured, for example, in a triangular, or crown-shaped pattern. Support edges 1006, 1007 may form the top and/or bottom ends of the elongated cells 1004.

Each of the cells 1004 may be defined by support edges 1006, 1007 and elongate connectors 1005 extending therebetween. Each elongate connector 1005 may extend from one of the cells 1003 to the support edge 1007 at the second end 1000b. The enlarged cells 1004 may be designed to reduce the amount of foreign material positioned against the aortic wall that may lead to undesired endothelialization. An open area of an enlarged cell 1004 may be at least 5×, at least 7×, or at least 10× greater than an open area of one of the cells 1003. As illustrated, there are three elongate connectors 1005 evenly spaced apart around a circumference of the frame 1010, with three cells 1004 positioned around the circumference of the frame 1010. But it is envisioned that there may be a fewer or greater number of connectors 1005 or cells 1004.

In some embodiments, the cover 1008 overlays a portion of the circumference of the blood flow modification device 1000, restricting radial expansion of the device in the covered regions, and uncovered portions are configured to allow for radial device expansion. A sufficient amount of the circumference of the device surface remains uncovered by the frame cover to increase radial expansion of the device by approximately 7 mm to 25 mm. Device expansion advantageously enables the device to adjustably conform to inherent inconsistencies of the inner diameter of a body lumen at the deployment site and/or reduces the number of device sizes required to accommodate body lumen size variations in the patient population.

In some embodiments, the cover 1008 may overlay, for example, approximately 40% to 90%, or 50% to 90%, or 50% to 80%, or 60% to 80%, or approximately one-half to three-fourths, or two-thirds, of the device circumference. The corresponding uncovered portion of the device is unconstrained by the frame cover and may be fully expandable when deployed. Examples of blood flow modification devices, and components thereof, suitable for use herein are described in Appendix A, attached herein and incorporated in its entirety.

At least a circumferential portion of the second section 1002 may remain uncovered to facilitate expansion of the second section 1002. If the cover 1008 were to surround the complete circumference of the second section 1002, the cover 1008 may limit expansion of the second section 1002. Leaving a portion of the second section 1002 uncovered also limits the amount of foreign material in contact with aortic wall in areas where endothelialization may not be desired.

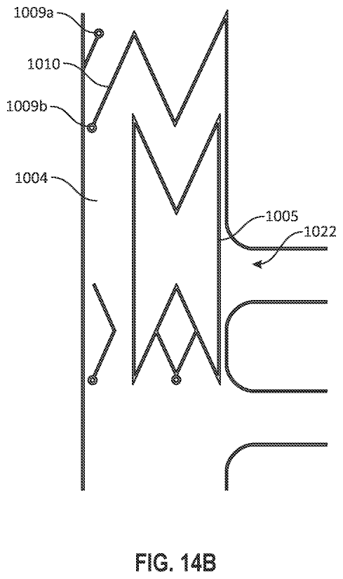

In some embodiments, all of the connectors 1005 may be used to support the cover 1008. For example, the cover 1008 may extend from a first strut 1005a to a second strut 1005b. Any additional connectors 1005 may be positioned between the first strut 1005a and the second strut 1005b to support the cover 1008. The uncovered portion of the section 1002 may not include any connectors 1005 such that an open cell 1004 of the second section 1002 is generally positioned opposite the ostium of the target vessel, as shown in FIG. 14B. This frame structure 1010 also limits the amount of foreign material in contact with the aortic wall in areas where endothelialization may not be desired.

Moreover, one or more visual indicators or radiopaque markers 1009 that are detectable, for example, by fluoroscopy may be located on one or more areas of the blood flow modification device 1000. For example, an eyelet formed on a bottom end of an elongated connector 1005 may be filled with radiopaque material, such as a gold-containing material or a tantalum-containing material, or other material that is sufficiently visible by fluorescence within the body of a patient. A radiopaque (RO) marker may be placed on the top 1000a and/or bottom 1000b of the device 1000, as top and/or bottom indicators, or between the first section 1001 and second section 1002.

The first section 1001 of the device may be the first portion of the device 1000 to be unsheathed during a deployment process. With the first section 1001 unconstrained by a cover 1008, the first section 1001 is free to fully expand when deployed outside of the delivery system. Under fluoroscopy a physician may visualize an indicator 109a, 109c in the first section 1001 to confirm delivery position relative to a target body lumen, such as the celiac artery, before deploying the second section 1002. A bottom indicator 1009 may be used to confirm that the ostium of an adjacent artery, such as the SMA, is not covered by the device 1000. The bottom indicator 1009 may be visible while the device 1000 is still within the delivery system. Optionally, where the cover 1008 overlays only a portion of the frame circumference, one or more alignment indicators 1009b may be placed on a frame strut opposite the cover and between top and bottom indicators. Using these indicators, the physician may identify the position of the mesh cover prior to full deployment. Where fluorescence detection is not sufficiently visible to accurately rotationally align the mesh, the external alignment feature of the delivery system is provided.

As shown in FIG. 12B, the frame 1010 may include one or more markers 1009. The markers 1009 may be positioned within eyelets or otherwise joined to the frame 1010. The one or more markers 1009 may be positioned in the first section 1001 and/or the second section 1002 of the frame 1010.

The first section 1001 may include a first set of markers 1009 configured to provide an indication of rotational alignment relative to the ostium. The first set of markers 1009 may be positioned at a first end 1000a of the frame 1010 and/or at the interface between the first section 1001 and the second section 1002. The frame 1010 may include a first marker 1009a at the first end 1000a of the frame 1010. The first marker 1009a may be configured to be positioned generally opposite cover 1008 and/or the ostium of the target vessel. The first marker 1009a may be rotationally offset from any of the elongate connectors 1005. The frame 1010 may include a second marker 1009b at the interface between the first section 1001 and the second section 1002. The second marker 1009b may be configured to be positioned generally opposite the cover 1008 and/or the ostium of the target vessel. The second marker 1009b may be rotationally offset from any of the elongate connectors 1005. The frame 1010 may include a third marker 1009c positioned at the first end of the frame 1010. The third marker 1009c may be aligned with one of the elongate connectors 1005. The third marker 1009c may be configured to be positioned near cover 1008 and/or the ostium of the target vessel. After the first section 1001 has been deployed, the physician may use the markers 1009 in the first section 1001 to locate the position of the cover 1008 and confirm rotational alignment of the device 1000. The physician may confirm rotational alignment prior to releasing the second section 1002 of the device 1000.

The second section 1002 may have one or more markers 1009 at the second end 1000b of the frame 1010 to provide an indication of axial position. As explained above, it may be desirable for the cover 1008 to only be placed against a single ostium. The second set of markers 1009 provides a confirmation that the device 1000 is not covering another vessel that may be necessary to perfuse the gastrointestinal organ. The one or more markers 1000 may be equally spaced apart around the second end 1000b of the frame 1010. Each of the markers 1009 in the second section 1002 may be rotationally offset from the elongate connectors 1005. For example, each marker 1009 in the second section 1002 may be positioned between two adjacent elongate connectors 1005. The second section 1002 may include the same number of markers 1009 as elongate connectors 1005.

As shown in FIG. 12B, the frame 1010 may include one or more attachment points 1015 for attaching the cover 1008. The attachment points 1015 may be eyelets. The cover 1008 may be secured to the one or more attachment points 1015 by sutures.

The frame 1010 may include a first set of attachment points 1015 at an interface between the first section 1001 and the second section 1002. At least some of the cells 1003 in the first section 1001 that are not joined to the elongate connectors 1005 may have an attachment point 1009 at the interface between the first section 1001 and the second section 1002. Cells 1003 in the first section 1001 that are not rotationally aligned with the cover 1008 may not have any attachment points 1015.

The frame 1010 may include a second set of attachment points 1015 at the second end 1000b of the frame 1010. For example, the second end 1000b of the frame 1010 may include alternating markers 1009 and attachment points 1015. Each of the attachment points 1015 may be aligned with one of the elongate connectors 1005.

FIG. 14A illustrates the axial position of the device 1000 relative to the target artery. In this photograph, the second section 1002 of the device 1000 is positioned across the ostium of the celiac artery to promote occlusion. The first section 1001 of the device 1000 is positioned beyond the ostium to anchor the device 1000. The second end of the device is positioned between the celiac artery and the superior mesenteric artery and does not obstruct flow through the superior mesenteric artery.

FIG. 14B illustrates a cross-section showing the position of the device 1000 relative to the ostium 1022. As illustrated, the open cell 1004 is positioned opposite the ostium 1022. The markers 1009a, 1009b in the first section 1001 may be used to confirm the rotational position of the cover 1008 relative to the ostium. The markers 1009a, 1009b are designed to be positioned opposite the cover 1008 and the ostium 1022. The additional markers 1009 at the second end 1000b of the device 1000 may be used to confirm that the device 1000 does not cover any arteries other than the target artery.

### Delivery System

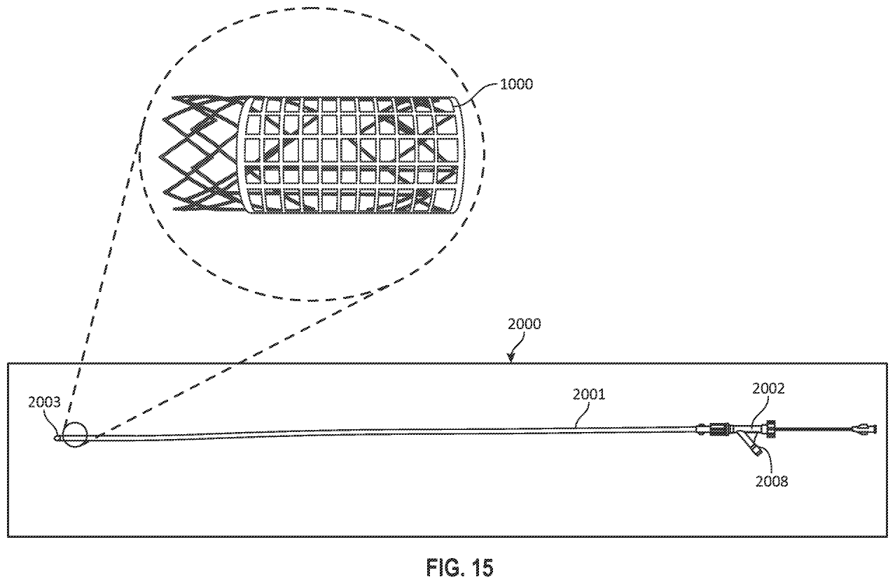

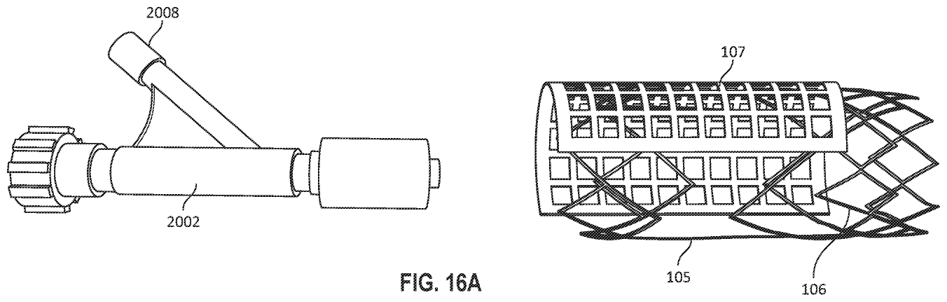

A system is described for delivering and positioning a blood flow modification device within a body lumen of a patient. With reference to FIG. 15, a delivery system 2000 is described that comprises an external rotational alignment element 2002 that aligns the blood flow modification device 1000 deployed within the body lumen. The alignment element 2002 is external to the body of a patient when the delivery system 2000 is in use. A method is described for deploying the blood flow modification device 1000 in a body lumen and rotationally aligning the device 1000 deployed in the body lumen by referencing the position of the external alignment element 2002. A further method is provided for partially occluding an artery to reduce the flow of blood to a gastric organ serviced by the artery, to promote weight loss. The blood flow modification device 1000 may be a stent, such as a bariatric stent, comprising a mesh cover.

Exemplified in FIG. 15, a blood flow modification device delivery system 2000 is illustrated having an external rotational alignment element 2002. The delivery system 2000 comprises a shaft 2001 connected to rotational alignment element 2002, which may be a hemostasis valve “Y” connector, at a proximal end and a tip 2003 at the distal end. The hemostasis valve “Y” connector held by the physician during use, is fixed in place to prevent rotational movement of the alignment element around the longitudinal axis of the delivery system 2000, forming the external rotational alignment element 2002. An adhesive may be dispensed to a rotating junction of the connector to restrict rotation of the hemostasis valve “Y” connector. A pin pull delivery system for deploying the device is illustrated.

In embodiments where the blood flow modification device frame 1010 is only partially covered by the frame cover 1008, the rotational alignment element 2002 may confirm alignment of the frame cover 1008 over the ostium of a target blood vessel.

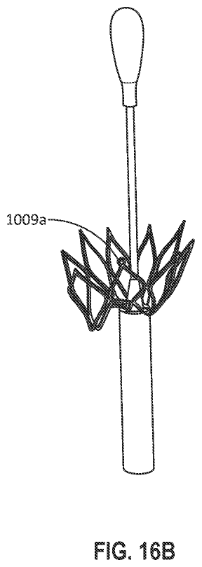

The blood flow modification device 1000 may be loaded into the delivery system 2000 and configured so that the frame cover 1008 and indicator 2008, e.g., “Y” port, of the hemostasis valve that has been fixed to prevent rotation of the connector around the longitudinal axis of the delivery device are in alignment. For example, at least a portion of the frame cover 1008 and the rotationally fixed indicator 2008 may both face upwardly (e.g., FIG. 16A), relative to the longitudinal axis of the deployment device 2000. Before deploying the blood flow modification device 1000 into a first body lumen, the indicator 2008 may be aligned with a second, target body lumen, thereby aligning the cover (e.g., mesh graft) with the ostium of the second, target body lumen when deployed.

The delivery system and devices described herein may be used to reduce blood flow to a gastric organ to promote weight loss. A method is provided for positioning a blood flow modification device 1000 in a first blood vessel to modify blood flow through a second blood vessel. Modification of blood flow through a second blood vessel may be selected to induce mesenteric ischemia, impeding normal gastrointestinal functioning to induce weight loss. When placed in a first artery adjacent the ostium of a second artery, the device 1000 may gradually reduce blood flow through the second artery that supplies to an organ of the gastrointestinal tract while collateral blood flow to the organ is established. The second artery may comprise the celiac artery resulting in perfusion of one or more of the liver, spleen, stomach or pancreas, or the superior mesenteric artery, or one or more of the branches thereof.

A method of deploying the blood flow modification device 1000 comprises one or more of the following steps. The method may include obtaining a delivery system 2000 that comprises a pre-loaded blood flow modification device 1000 with a frame cover 1008. The frame cover 1008 may be in alignment with a rotationally fixed, external rotation alignment element 2002.

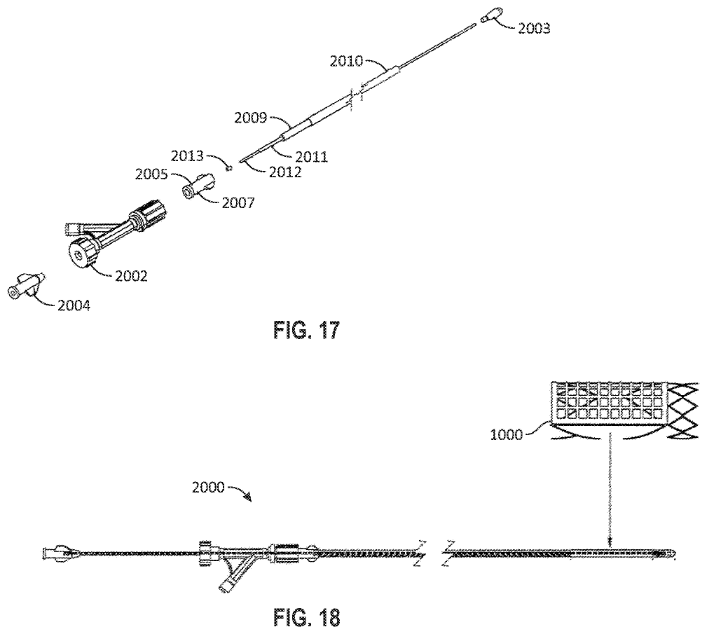

The delivery system 2000 may be advanced over a guidewire (e.g., 0.035″) and/or through a sheath in the femoral artery positioning the tip above the target artery, for example the celiac artery. The method may include partially unsheathing the outer sheath 2010, shown in FIG. 17, to align the first section 1001 of the blood flow modification device 1000 (e.g. having a top visual alignment indicator 1009a) with the sheath tip. The sheath tip may have a radiopaque marker. The delivery system 2000 may be advanced or retracted to align bottom device indicators 1009 above the SMA or to otherwise not block any arteries other than the target artery. The method may include retracting the sheath 2010 and deploying the first section 1001 of the device 1000 and top visual alignment indicators 1009a, as shown in FIG. 16B. The method may include rotating the delivery system 2000 so that the indicator 2008 is aligned with the target artery. The method may include unsheathing until the first section 1001 of the blood flow control device (without a cover) deploys and the struts of the device frame expand (FIG. 16B). Optionally, the method may include confirming alignment indicators 1009a, 1009b are positioned opposite the celiac artery ostium or rotating to achieve alignment. After rotational alignment has been confirmed, the method may include unsheathing until device 1000 is fully deployed. The catheter tip 2003 may be retracted into delivery system outer sheath and removed from the body.

Further drawings of the delivery system 2000 are exemplified in FIGS. 17-18, wherein device components include a stent subassembly 1000, catheter tip 2003, outer sheath 2010, outer sheath hub 2007, middle tubing 2009, hypotube 2011, hypotube hub 2004, guidewire lumen 2012, rotational alignment element 2002, adhesive 2005, and/or heat shrink 2013.

The dimensions of the blood flow modification device 1000 may be sufficiently sized to be retained within a range of artery sizes; for example, the device 1000 may form a tube having an outer diameter that may expand (before over expansion) to a diameter between 2 mm and 30 mm, or between 7 mm and 25 mm, or between 10 mm and 30 mm, such as 13 mm to 30 mm, such as approximately 21 mm, or between 2 mm to 10 mm, such as between 2 mm to 5 mm. The length of the device 1000 in a deployed state may be determined by the target location, wherein the length of a top section is sufficient to hold the device in place within the artery, and the length of the lower section is sufficiently long to cover an ostium of a branching artery without blocking any adjacent branching arteries. For example, in some embodiments, the total device length may be 50 mm or greater, such as between 50 mm and 55 mm, for example 54 mm, or the length of the device may be 50 mm or less, such as between 30 mm and 50 mm, or between 20 mm and 30 mm, or between 10 mm and 30 mm,

A method of inducing weight loss is provided comprising deploying a blood flow modification device 1000 within a first artery and positioning the frame cover adjacent the ostium of a second target artery that supplies blood to an organ of the gastrointestinal tract by confirming and/or rotating the rotational alignment indicator 2008, e.g., Y port of the delivery system, to align with the target artery ostium. The method may further include gradually reducing blood flow through the target artery as endothelial growth decreases the size of openings of a portion of the device cover 1008 overlaying the target artery ostium, thereby restricting blood flow to one or more organs and inducing weight loss. Hypoperfusion of the stomach or other gastrointestinal organ may be effected by modification of one or more target blood vessel of the gastrointestinal tract including the celiac artery or superior mesenteric artery; in other embodiments, the blood vessel may be the left gastric artery, the right gastric artery, the left gastroepiploic artery, the right gastroepiploic artery or the common hepatic artery, the inferior pancreaticoduodenal artery, jejunal and/or ileal arteries, or a combination thereof.

### Terminology

As used herein, the relative terms “top” and “bottom” shall be defined from the perspective of arterial blood flow. Thus, top refers to a direction closer to the heart and bottom refers to a direction further from the heart.

As used herein, the relative terms “proximal” and “distal” with respect to the device shall be defined from the perspective of arterial blood flow. Thus, proximal refers to a direction closer to the heart and distal refers to a direction further from the heart.

As used herein, the relative terms “proximal” and “distal” with respect to the delivery system shall be defined from the perspective of the delivery system. Thus, proximal refers to a direction closer to the handle and distal refers to a direction closer to the tip.

From the foregoing description, it will be appreciated that an inventive device and approaches for using a blood flow modification device are disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations can also be implemented in combination in a single implementation. Conversely, various features that are described in the context of a single implementation can also be implemented in multiple implementations, separately, or in any suitable sub-combination. Moreover, although features may be described above as acting in certain combinations, one or more features from a claimed combination can, in some cases, be excised from the combination, and the combination may be claimed as any sub-combination or variation of any sub-combination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of, the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily convey an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A blood flow modification device for inducing weight loss, the device comprising:
a frame comprising:
a first section at a first end of the frame, the first section being uncovered and configured to anchor the blood flow modification device in a first vessel, the first section comprising a first cell pattern;
a second section extending from the first section to a second end of the frame, the second section supporting a porous cover configured to be positioned across an ostium of a second vessel, the porous cover only partially surrounding a circumference of the frame, the second section comprising a second cell pattern different from the first cell pattern, the second section further comprising a plurality of elongate struts circumferentially spaced apart, each of the plurality of struts extending from the first section to the second end of the frame.

2. The device of claim 1, wherein the first cell pattern comprises a first cell size, and wherein the second cell pattern comprises a second cell size larger than the first size.

3. The device of claim 1, wherein the plurality of elongate struts is three elongate struts.

4. The device of claim 1, wherein the cover extends from one of the plurality of elongate struts to another one of the plurality of elongate struts.

5. The device of claim 1, further comprising a plurality of radiopaque markers at the second end of the frame.

6. The device of claim 5, wherein the plurality of radiopaque markers are circumferentially offset from the plurality of elongate struts.

7. The device of claim 1, wherein the first section comprises a radiopaque marker configured to be positioned generally opposite the ostium of the second vessel.

8. The device of claim 7, wherein the radiopaque marker is positioned at the first end of the frame.

9. The device of claim 7, wherein the radiopaque marker positioned at an interface between the first section and the second section of the frame.

10. The device of claim 1, further comprising a plurality of anchors for attaching the cover.

11. The device of claim 10, wherein at least one of the plurality of anchors is positioned at the second end of the frame.

12. The device of claim 11, wherein each of the at least one of the plurality of anchors at the second end of the frame is aligned with one of the plurality of elongate struts.

13. The device of claim 10, wherein at least one of the plurality of anchors is positioned between the first section and the second section.

14. A blood flow modification device for inducing weight loss, the device comprising:
a frame comprising:
a first section at a first end of the frame, the first section being uncovered and configured to anchor the blood flow modification device in a first vessel;
a second section supporting a porous cover configured to be positioned across an ostium of a second vessel, the cover only partially surrounds a circumference of the frame,
wherein the cover extends from the first section to a second end of the frame.

15. The device of claim 14, wherein the cover comprises a uniform porosity.

16. The device of claim 14, wherein the second section comprises a plurality of elongate struts extending from the first section to the second end of the frame.

17. The device of claim 16, wherein the plurality of elongate struts is three elongate struts.

18. The device of claim 14, further comprising a plurality of radiopaque markers at the first end and/or the second end of the frame.

19. The device of claim 14, further comprising a plurality of anchors for attaching the cover.

20. The device of claim 19, wherein a first set of the plurality of anchors is positioned between the first section and the second section of the frame.

21. The device of claim 20, wherein a second set of the plurality of anchors is positioned at the second end of the frame.

22. A blood flow modification device for reversibly decreasing blood flow through an artery, the device comprising:
a top section having a first cell pattern, and
an adjacent lower section comprising a second cell pattern comprising
at least two elongated cells,
a plurality of elongated connectors, and
a support edge at a bottom end of the lower section connected to the elongated connectors,
wherein the first cell pattern has a cell density greater than the second cell pattern; and
a cover over at least a portion of the lower section,
wherein the device is configured to expand within a first artery to cover an ostium of a second artery.

23. The blood flow modification device of claim 22, wherein the device is in the shape of an expandable tube.

24. The blood flow modification device of claim 22, wherein the elongated connectors extend the length of the lower section.

25. The blood flow modification device of claim 22, wherein the device comprises a shape recoverable material.

26. The blood flow modification device of claim 22, wherein the elongated connectors comprise a metal alloy.

27. The blood flow modification device of claim 22, wherein the cell of the lower section is formed between adjacent elongated connectors.

28. The blood flow modification device of claim 22, wherein the lower section comprises a radiopaque marker on at least one elongated connector opposite the cell configured to cover the ostium.

29. The blood flow modification device of claim 22, wherein the cover overlays the cell and partially occludes the ostium of the second artery.

30. The blood flow modification device of claim 22, wherein the cover comprises a fabric.

31. The blood flow modification device of claim 22, wherein the cover comprises a polyester.

32. The blood flow modification device of claim 22, wherein the covers supports endothelial cell growth.

33. The blood flow modification device of claim 22, wherein the covers comprises openings that reduce blood flow rate.

34. A method of inducing weight loss in a patient, the method comprising:
advancing a delivery system carrying a blood flow modification device to an abdominal aorta, the blood flow modification device comprising a frame carrying a cover that only partially surrounds a circumference of the frame;
rotationally aligning the cover with an ostium of a celiac artery;
deploying the blood flow modification device in the abdominal aorta such that the cover is positioned across the ostium of the celiac artery; and
gradually occluding at least partial blood flow through the ostium of the celiac artery.

35. The method of claim 34, further comprising partially deploying the blood flow modification device.

36. The method of claim 35, further comprising confirming a radiopaque marker on the partially deployed section of the blood flow modification device is positioned opposite the ostium of the celiac artery.

37. The method of claim 34, wherein deploying the blood flow modification device comprises unsheathing the blood flow modification device.

38. The method of claim 34, wherein deploying the blood flow modification device comprises positioning an end of the blood flow modification device between the celiac artery and a superior mesenteric artery.

39. The method of claim 34, wherein gradually occluding at least partial blood flow through the ostium of the celiac artery does not fully occlude blood flow through the ostium of the celiac artery.

40. The method of claim 34, wherein gradually occluding at least partial blood flow through the ostium of the celiac artery comprises occluding flow by at least 60% within one month.

41. The method of claim 34, wherein after gradually occluding at least partial blood flow through the ostium of the celiac artery, regaining access to the celiac artery by advancing an instrument through the cover and the ostium of the celiac artery.

42. The method of claim 41, wherein regaining access to the celiac artery comprises advancing the instrument to the abdominal aorta and through the cover to the celiac artery.

43. The method of claim 41, wherein gaining access to the target artery comprises advancing the instrument through a superior mesenteric artery and into the celiac artery and then through the cover into the abdominal aorta.

44. The method of claim 34, further comprising confirming rotational alignment of the cover with the ostium of the celiac artery using an external rotational alignment element on the delivery system.

45. The method of claim 44, further comprising rotating the delivery system until the external rotational alignment element is aligned with the ostium of the celiac artery.

46. A method of inducing weight loss comprises the steps of:
obtaining a delivery system comprising a blood flow modification device and deploying a blood flow modification device within a first artery,
wherein the delivery system comprises an external rotational alignment element,
wherein the blood flow modification device comprises a frame and a frame cover that partially surrounds the frame circumference, and
wherein the frame cover and the external rotational alignment element are aligned within the delivery system; and
positioning the frame cover adjacent the ostium of a second target artery that supplies blood to an organ of the gastrointestinal tract by rotating the delivery system so that the external rotational alignment element is aligned with the target artery ostium.

47. The method of claim 46, wherein the external rotational alignment element comprises a hemostasis valve “Y” comprising a Y port that is aligned with the frame cover in the delivery system, and wherein the hemostasis valve “Y” connector is in a rotationally locked position.
